(19) United States
(12) Patent Application Publication
(10) Pub. No.: US 2021/0252050 A1
(43) Pub. Date: Aug. 19, 2021

(54) ANTI-INFLAMMATORY, DISINFECTING AND ACCELERATED HEALING GEL FOR WOUNDS AND LESIONS

(71) Applicant: NANOCARE PHARMA INC., San Marcos, TX (US)

(72) Inventors: David L. COCKE, San Marcos, TX (US); Gary Wayne BEALL, San Marcos, TX (US)

(21) Appl. No.: 17/155,748

(22) Filed: Jan. 22, 2021

Related U.S. Application Data
(60) Provisional application No. 62/978,632, filed on Feb. 19, 2020.

(51) Int. Cl. A61K 33/40; A61K 47/02; A61K 9/06; A61P 17/02

(52) U.S. Cl. CPC A61K 33/40; A61P 17/02; A61K 9/06; A61K 47/02

(57) ABSTRACT

A composition and process for use in wound healing and a process for making such a composition has a smectite gelling agent free of heavy and translon metals mixed with hydrogen peroxide and with water. The smectite gelling agent is a synthetic aluminosilicate having the formula (Na)ₓ(Al₂—XMgₓ)2Si₂O₁₀(OH)₂.n(H₂O) or Naₓ(Mg₃—XLiₓ)3Si₄O₁₀(OH)₂ where X is between 0.1 and 0.5. The smectite gelling agent has a concentration between 1-7% w/w of the total composition. The hydrogen peroxide has a concentration of 1-6% w/w of the total composition. The smectite gelling agent, the hydrogen peroxide and water are mixed together in a mixer.

### CROSS-REFERENCE TO RELATED APPLICATIONS

The present application claims priority from U.S. Provisional Patent Application Ser. No. 62/978,632, filed on Feb. 19, 2020 and entitled “Anti-Inflammatory, Disinfecting and Accelerated Healing Gel for Wound and Lesions”.

### STATEMENT REGARDING FEDERALLY SPONSORED RESEARCH OR DEVELOPMENT

Not applicable.

### NAMES OF THE PARTIES TO A JOINT RESEARCH AGREEMENT Not applicable.

### INCORPORATION-BY-REFERENCE OF MATERIALS SUBMITTED ON A COMPACT DISC

Not applicable.

### BACKGROUND OF THE INVENTION

### 1. Field of the Invention

The present invention relates to the treatment and healing of wounds. More particularly, the present invention the relates to compositions used for the healing of such wounds.

### 2. Description of Related Art Including Information Disclosed Under 37 CFR 1.97 and 37 CFR 1.98

It is well-known that hydrogen peroxide is an effective disinfecting agent for use in wound care and oral applications. Hydrogen peroxide is also widely used for effectively eliminating biofilms found in extremely difficult infections, including antibiotic-resistant bacteria. There are many different compositions utilizing hydrogen peroxide for disinfecting surgical equipment, hard surfaces, toilets, drinking water, and many other applications. It is well understood that the disinfecting effect of hydrogen peroxide takes substantial time to kill bacteria and fungi. This requires the hydrogen peroxide to be in contact with the bacteria or fungi for an extended period of time. This normally is accomplished by soaking the contaminated area in an aqueous solution of the hydrogen peroxide or by spraying and wiping the surface with the solution and allowing it to evaporate or by soaking the cloth in the hydrogen peroxide and then applying the soaked cloth in place long enough to disinfect. All of these methods are inconvenient. For most wounds, simple irrigation does not allow enough time for the hydrogen peroxide to be effective.

One method that overcomes many of the problems associated with applying hydrogen peroxide is to form a gel that will stay in place. These gels have been utilized to bleach hair, whiten teeth, disinfect surfaces, and rinse orally. There are a number of synthetic and natural polymers used that will form a stable gel with the hydrogen peroxide. These gelling agents are effective at keeping the hydrogen peroxide in place but suffer when forming a sticky film since the water evaporates. Such gelling agents are not pleasant to the touch and can also stain clothing. The coating will also build up on surfaces after repeated use. Hydrogen peroxide is also an effective and extremely reactive species and oxidizes most of the polymer reagents, decomposing the hydrogen peroxide, along with its disinfectant properties. Smectite clays, a common gelling agent in topical and cosmetic solutions, would be a better choice as a thickener. However, all natural and most natural and synthetic smectites contain substantial amounts of transition metal contaminants that catalyze the decomposition of hydrogen peroxide.

In the medical field, various hydrogen peroxide-containing compositions have been utilized to disinfect implants prior to implantation or as bandages for burns or other injuries to the skin. In the case of direct application of hydrogen peroxide to burns or other injuries to the skin, the concentration of the hydrogen peroxide is kept below 0.1%. The reason for this low concentration of hydrogen peroxide is that medical literature clearly recommends not putting more than trace amounts of hydrogen peroxide in a wound because it will kill good tissue and retard healing. As such, it is desirable to produce a hydrogen peroxide gel that is stable upon storage, does not leave a tacky residue, and actually disinfects while promoting healing.

The first hydrogels were made in the mid-1900s by using water absorbent and cross-linked polymers that increase the viscosity of the aqueous solution. These hydrophilic polymers retained water while also sustaining the cross-link structure so as to give structural support and turn a liquid into a gel-like medium. The three-dimensional cross-link of these hydrophilic polymers interact with water and create this gel state by physical and chemical forces, including hydrogen bonding, mechanical chain entanglement, and hydrophobic interactions. Of course, many other forces are involved depending on the nature of the polymer and any of the other ingredients in the hydrogels.

Hydrogels can be characterized in the three groups which include physical, chemical and biochemical. These categories are defined by the process which causes the liquid to turn into the gel state. Physical gels are created by changing physical properties of the solution, such as temperature, pressure and added radiation. For example, long polylactic acid cross-links are used to form a hydrogel by modifying temperature during synthesis similar to agarose that gels as the temperature cools. Chemical gels are synthesized by altering properties such as pH, intermolecular interactions, and chemical/ionic compositions of the solution. Chemical gels are most commonly made by changing covalent bonding between crosslinks and solvents, such as HA-az-F127 hydrogel. A very new kind of hydrogel incorporates biochemical components, such as enzymes and amino acids, in the gelation process. These kinds of hydrogels are becoming increasingly common, but different gelling agents are used. Clays, such as smectite clays, serve as a better gelling agent replacement because of their innate plate like morphology, non-toxic and inert nature. These smectite clays having a surface area of 750 m²/g are charge suitable for altering its hydrophobic-hydrophilic balance at will. It has a refractive index similar to polymer so that the nanoparticles appear transparent. These clays are ubiquitous around the world and are easily mined and purified. They originate from volcanic ash which has settled and transformed over millions of years into smectite by natural processes, such as isomorphic substitution of the crystal structure, thereby turning it into hectorite, montmorillonite, saponite, suconite, volchonskoite, vermiculite and nontronite. The composition of these clays can be visualized by imagining a sandwich structure with the top and bottom layers composed of silica dioxide tetrahedral structures. The center layer is composed of a transition metal oxide octahedral layer. This transition metal in the octahedral structure is what is isomorphicly substituted with an uncharged layer of all al⁺³, which makes it a dioctahedral structure, to a negatively charged layer with Mg²⁺, which makes it a trioctahedral structure producing smectite clay. The amount of substitution in the sheets will dictate the strength of charge in the nano particle. However, nature will not abide a net charge development without supplying countrions such as sodium, calcium, potassium, etc. ions. The combination of water-soluble counter ions, such as sodium, plus the substitution is what gives smack tight clays there water solubility so as to allow full exfoliation when and dispersion while in solution. Isomorphic substitution can also occur in the tetrahedral silica dioxide layer with Al substituting for is. These smectite clays, such as LAPONITE™, can also be made synthetically at high pressures and high temperatures so as to ensure chemical purity by eliminating transition metals while also retaining water solubility.

More recently, clays have been used in more topical solutions and hydrogels because of their innate and benign properties. Unlike some hyperallergenic synthetic polymers, smectite clays are environmentally ubiquitous and are found in soil, food and even ambient air. This makes it one of the safest and benign substances on earth.

In the past, various patents and patent application publications have issued with respect to the field of wound treatment. For example, U.S. Pat. No. 8,202,532, issued on Jun. 19, 2012 to Huey et al., teaches a clay-based hemostatic agent and devices for the delivery thereof. These are used for promoting the clotting of blood. The agent is comprised of a clay material and a release agent. The clay material is disposed within a substrate and the release agent is disposed within a mesh. The release agent can be configured to make direct contact with a bleeding wound when the device is in particle form and the clay material can promote the hemostasis.

U.S. Pat. No. 9,302,025, issued on Apr. 5, 2019 to Baker et al., describes a hemostatic composition comprising a wet-layered clay (e.g. wet kaolin) and, optionally, a zeolite. These devices are used for promoting blood clotting.

U.S. Pat. No. 9,585,820, issued on Mar. 7, 2017 to Lull et al., describes a laponite clay for use in cosmetic and personal care products. In particular, a multi-component composition includes a first liquid composition comprising water, a laponite clay and a first component, and a second liquid composition comprising water, a polymer thickener and a second component. The first and second components are separated such that the interaction between them is reduced compared to an otherwise identical multi-component composition lacking the laponite clay. The first and second components are chemically incompatible.

U.S. Pat. No. 9,744,238, issued on Aug. 29, 2017 to Supamahitorn et al., describes a high-load dispersion in the field of carrier and delivery systems for active molecular compounds. In particular, this patent describes a method for disinfecting a surface in which an antimicrobial composition is prepared by dry mixing a layered hydrous magnesium silicate clay and a quaternary ammonium compound to produce a mixture, adding water to the mixture to form a suspension, and incubating the suspension in the water to form an antimicrobial composition. The quaternary ammonium compound is loaded into the layered hydrous magnesium silicate clay.

U.S. Patent Application Publication No. 2007/0134293, published on Jun. 14, 2007 to Huey et al., discloses an apparatus for promoting the clotting of blood. This apparatus comprises a receptacle in which at least a portion is defined by a mesh having openings therein, and particles of clay retained in the receptacle. First and second blood clotting materials are enclosed in the mesh. The mesh structure is placed on a bleeding wound such that the second blood clotting material contacts wounded tissue of the bleeding wound.

U.S. Patent Application Publication No. 2017/0202877, published on Jun. 20, 2017 to Hoover et al., teaches a formulation for use in wound healing. The formulation includes hypochlorous acid, a silicone polymer, sodium phosphate, hydrochloric acid, and sodium magnesium silicate.

U.S. Patent Application Publication No. 2020/0179297, published on Jun. 10, 2020 to El-Sabahy et al., teaches a nanotechnology-based hemostatic dressing. This hemostatic composition includes a combination of more than one hemostatic agent and devices coated or impregnated therewith. The nano-hemostatic agents have large surface areas so as to increase the hemostatic properties when they were applied. By combining more than one hemostatic agent and utilizing one or more different nanotechnology approaches to enhance the surface areas thereof, the capability of the dressing to stop bleeding is improved by more than one mechanism so as to provide better hemostasis.

International Publication No. WO 2012/084605, published on Jun. 28, 2012 to Lesch et al., discloses a leave-on composition having laponite and an essential oil. In particular, the composition comprises in a physiologically acceptable medium of at least one laponite, at least one essential oil, and at least one additional hydrophilic gelling agent. The hydrophilic gelling agent is chosen from gelling agents of natural origins, polysaccharides of biotechnological origin, and mixtures thereof. It is used for preventively and/or curatively treating greasy skin or acne-prone skin.

U.S. Patent Application Publication No. 2005/0129782, published on Jun. 16, 2005 to Ketelson et al., describes the use of inorganic nanoparticles to stabilize hydrogen peroxide solutions. The nanoparticles are formed from clays. The use of nanoparticles from synthetic smectite clays is particularly preferred. The stabilized hydrogen peroxide solutions can be utilized for various purposes such as the disinfecting of contact lenses.

It is an object of the present invention to provide a composition and process that promotes the healing of wounds.

It is another object of the present invention to provide a composition and process that inhibits the cascade of painful inflammations.

It is another object of the present invention to provide a composition and process that is in a gel form.

It is another object of the present invention to provide a composition and process that is stable upon storage.

It is another object of the present invention to provide a composition and process that does not have tacky residue.

It is another object of the present invention to provide a composition and process that disinfects while promoting healing.

It is another object of the present invention provide a composition and process that is safe and benign.

It is another object of the present invention to provide a composition and process that accelerates the transition of fibroblasts to myofibroblasts.

It is still a further object of the present invention to provide a composition and process that is free of heavy metals.

These and other objects and advantages of the present invention will become apparent from a reading of the attached specification and appended claims.

### BRIEF SUMMARY OF THE INVENTION

The present invention is address the problems associated with hydrogen peroxide compositions for treating and disinfecting superficial abrasions, lacerations, scrapes and chronic wounds, along with fungal infections, such as athletes' foot and nail fungus. Unexpectedly, the present invention actually promotes healing of wounds and inhibits the cascade of painful inflammations. The present invention is a gel and composed of dilute hydrogen peroxide and a synthetic magnesiosilicate clay (referred to as “LAPONITE”™). The synthetic magnesiosilicate clay is added to thicken the hydrogen peroxide into a gel. As the gel dries, the “LAPONITE”™) creates evenly dispersed trioctahedral nanosheets over the wound so as to provide structural support and an air-tight barrier over the wound. Since hydrogen peroxide is an extreme oxidizer, it has not been possible previously to add a gelling agent without decomposing the hydrogen peroxide, along with its disinfectant properties. “LAPONITE”™) is a new and synthetic bentonite clay which contains no transitional metals. The “LAPONITE”™) not only does not react with the hydrogen peroxide but also stabilizes it for exponentially longer periods of time than liquid peroxide. This allows the hydrogen peroxide to disinfect for a longer period of time. The “LAPONITE”™) also interacts with the body by accelerating transition of fibroblasts to myofibroblasts which initiate wound contraction. The transition not only accelerates the wound healing process, but also inhibits a natural cascade of natural and painful information.

The present invention is a process for use in wound healing in which the process includes the step of mixing a smectite gelling agent with hydrogen peroxide and water together in a mixer so as to form a gel. The mixture is free of heavy metals and the gel is free of heavy metals. The mixer can be a tumbling closed container, a rolling closed container, a cowl dissolver, a paddle mixer, a colloid mill, and ultrasonification mixture, or combinations thereof

The hydrogen peroxide is mixed with water prior to the step of mixing the smectite gelling agent with the hydrogen peroxide. The hydrogen peroxide is mixed 1-6% w/w with the water. In the preferred embodiment the present invention, the hydrogen peroxide is mixed between 2-5% w/w with the water.

The smectite gelling agent is a synthetic aluminosilicate having the formula (Na)ₓ(Al₂—XMgₓ)2Si₂O₁₀(OH)₂.n(H₂O) where X is between 0.1 and 0.5. Alternatively, the smectite gelling agent can be a synthetic alumina silicate having the formula Naₓ(Mg₃—XLiₓ)3Si₄O₁₀(OH)₂ where X is between 0.1 and 0.5. In the present invention, the hydroxyl group (OH) of either of the previously-described formulas can be substituted wholly or partially with fluoride ions.

In the present invention, the smectite gelling agent will have a concentration of between 1-7% w/w of the mixture. In the preferred embodiment of the present invention, the smectite gelling agent will have a concentration between 2-3% w/w of the mixture.

The present invention is also a composition for use in wound healing. This composition has a smectite gelling agent free of heavy metals, hydrogen peroxide and water mixed together so as to form a gel. The hydrogen peroxide is 1-6% w/w of the composition. Preferably, the hydrogen peroxide is 2-5% w/w of the total composition. The smectite gelling agent is a synthetic aluminosilicate having the formula (Na)ₓ(Al₂—XMgₓ)2Si₂O₁₀(OH)₂.n(H₂O) where X is between 0.1 and 0.5. Alternatively, the smectite gelling agent can be in a synthetic aluminosilicate having the formula Naₓ(Mg₃—XLiₓ)3Si₄O₁₀(OH)₂ where X is between 0.1 and 0.5. The hydroxyl group (OH) can be substituted wholly or partially with a fluoride ion in either of the above-describe synthetic aluminosilicates. The smectite gelling agent will have a concentration between 1-7% w/w of the total composition. In the preferred embodiment of the present invention, the smectite gelling agent will have a concentration of between 2-3% w/w of the total composition.

This foregoing Section is intended to describe, with particularity, the preferred embodiments of the present invention. It is understood that modifications to these preferred embodiments can be made within the scope of the appended claims without departing from the true spirt of the invention. As such, this Section should not to be construed, in any way, as limiting of the broad scope of the present invention. The present invention should only be limited by the following claims and their legal equivalents.

### DETAILED DESCRIPTION OF THE INVENTION

The present invention is a disinfecting gel that also unexpectedly promotes healing. The gel is an excellent disinfectant the can be used to treat fungal and bacterial infections. The gel is applied topically and, in the case of minor superficial wounds, eliminates the infection without internal medications which can have potential serious side effects on the liver. The most unexpected facet of the present invention is the promotion of healing when applied to a wound and its anti-inflammatory effects. Medical literature recommends against applying hydrogen peroxide to wounds as a disinfectant because it is cytotoxic to healthy cells as well as bacteria so that it slows healing. The gelled hydrogen peroxide of the present invention not only disinfects the wound but speeds up healing by three to four days, depending on the size of the wound, by skipping the inflammatory stage of healing. It also skips the inflammatory phase by initiating wound contraction over a twenty-four hour period. Usually wound contraction is initiated two weeks after the wound has formed. The phases of wound healing are (1) hemostasis; (2) inflammation; (3) proliferation or granulation; and (4) remodeling or maturation.

The process of hemostasis is similar to the circumstance where a source of damage to a house has been removed. Before work can start, utility workers must come in and cap damaged gas or water lines. Similarly, in wound healing, damaged blood vessels must be sealed. In wound healing, the platelet is the cell which acts as the utility worker sealing off the damaged blood vessels. The blood vessels themselves constrict in response to injury, but the spasm ultimately relaxes. The platelets secrete vasoconstrictive substances to aid in this process, but their prime role as to form a stable clot that seals the damaged vessel. Under the influence of adenosine diphosphate leaking from damaged tissue, the platelets aggregate and adhere to the exposed collagen. They also secrete factors which interact with and stimulate the intrinsic clotting cascade through the formation of thrombin which, in turn, initiates the formation of thrombin from fibrinogen. The fibrin mesh strengthens the platelet aggregate into a stable hemostatic plug. Finally, platelets also secrete cytokines, such as platelet-derived growth factor, which is recognized as one of the first factors secreted in initiating the subsequent steps. Hemostasis occurs within minutes of the initial injury, unless there are underline clotting disorders.

The inflammation phase is the second stage of wound healing. Clinically, inflammation is present as a eyrthema, swelling and warmth that is often associated with pain. This stage usually lasts up to four days post-injury. In the wound healing analogy, the first job to be done once the utilities are capped is to clean up the debris. This job is for non-skilled laborers. The non-skilled laborers in a wound are the neutrophils or polymorphonucleosytes. The inflammatory response causes the blood vessels to become leaky so as to release plasma and the polymorphonucleocytes into the surrounding tissue. The neutrophils phagocytize debris and microorganisms and provide the first line of defense against infection. They are aided by local mast cells. As fibrin is broken down as part of this clean-up, the degradation products attract the next cell involved. The next task of rebuilding a house is complex and requires someone to direct this activity or a contractor. The cell which acts as a “contractor” in wound healing is the macrophage. Macrophages are able to phagocytize bacteria and provide a second line of defense. They also secrete a variety of chemical chemotactic and growth factors, such as fibroblast growth factor, epidermal growth factor, transforming growth factor beta (TGF- and interleukin-1) which appears to direct the next stage.

The next step of wound healing is the proliferative phase that includes proliferation, granulation and contraction. The granulation stage starts approximately four days after wounding and usually lasts until day twenty-one in acute wounds (depending on the size of the wound). It is characterized clinically by the presence of pebbled red tissue in the wound base and involves replacement of dermal tissues and sometimes subdermal tissues in deeper wounds, as well as contraction of the wound. In the wound healing analogy, once the site has been cleared of debris, under the directions of the contractor, the framers move to build the framework of the new house. Subcontractors can now install new plumbing and wiring on the framework while siders and roofers can finish the exterior of the house. The “framer” cells are the fibroblasts which secrete the collagen framework and in which further dermal regeneration occurs. Specialized fibroblasts are responsible for wound contraction. The “plumber” cells are the parasites which regenerate the outer layers of capillaries and the endothelial cells so as to produce the lining. This process is called angiogenesis. The “roofer” and “sider” cells are the keratinocytes that are responsible for epithelialization. In the final stage of epithelialization, contracture occurs as the keratinocytes differentiate to form the protective outer layer or stratum corneum.

The final stage of wound healing is the remodeling or maturation phase. Once the basic structure of the house is completed, indoor finishing may begin. This is similar in wound healing in which repair involves remodeling the dermal tissues to produce greater tensile strength. The principle cell involved in this process is the fibroblasts. Remodeling can take up to two years after wounding and explains why apparently healed wounds can break down so dramatically and quickly if attention is not paid to the initial causative factors.

It is believed that the synthetic smectite clay is responsible for mediating the deleterious effects of hydrogen peroxide on unhealthy cells but must also sequester some of the signaling molecules that initiate the inflammation phase of healing. It further appears that as the water in the gel evaporates, the smectite clay forms a protective layer over the wound. This coating of clay also keeps the wound humidified due to the natural tendencies of smectites to absorb moisture from the air. In addition, the wound heals with less scarring such that the clay also plays a role in promoting the third and fourth stages of healing. This is due to the fact that when the wound skips the inflammation stage, less, trauma is experienced by the wound.

In experiments conducted on the present invention, new and unexpected results have been achieved. As such, this proves the efficacy of the present invention. The experiments conducted on the composition of the present invention are described hereinafter.

### EXAMPLE 1

In this example, the subject experienced chronic toenail fungus. The subject had previously attempted every treatment option available. The fungus had not been eliminated. The person started to apply the gel of the present invention which contained 3% w/w hydrogen peroxide and 2.5% smectite gelling agent to the nail. After five weeks, the healthy cuticle was restored and the diseased nail moved away from the cuticle.

### EXAMPLE 2

A second subject experienced athletes' foot periodically over many years. The athletes' foot outbreaks resulted in an itchy rash, bleeding cracks between the toes, and blisters on the soles of the foot. In the past, attempts to treat the malady with over-the-counter drugs would result in a cessation of symptoms in about three weeks. The subject applied the gel of the present invention containing 3% w/w hydrogen peroxide and 2.5% w/w smectite gelling agent twice daily. The symptoms disappeared in only three days.

### EXAMPLE 3

A third subject who was clearing brush encountered briers and received multiple scratches on their arms. The gel of the present invention containing 3% w/w hydrogen peroxide and 2.5% w/w smectite gelling agent was applied to only one arm while the other arm was left untreated as a control arm. The cuts on the control arm became red and inflamed and did not go into the second stage of healing for about three days. The control arm cuts required about two weeks to heal. The cuts treated with the gel of the present invention never became inflamed and began healing almost immediately. They healed in a week with almost no scarring. Similar healing of wounds has been experienced by dozens of individuals when employing the gel of the present invention.

### EXAMPLE 4

An individual was working on his barn and received a splitter in his finger. The subject thought that he had extracted all of the splitter but, the next day, the wound was inflamed and a small pustule had formed. A small amount of the gel of the present invention containing 3% w/w hydrogen peroxide and 2.5% w/w smectite gelling agent was applied to the wound. Within an hour the inflammation began to subside. The inflammation was completely gone the next day and the wound completely healed in four days.

In all of the examples referenced hereinabove, it was determined that more than 7% w/w of smectite gelling agent was incorporated into the composition, the composition was too viscous to apply and use. If less than 1% w/w smectite gelling agent was used, the composition became too liquid. It was so non-viscous that would be too thin to apply. When less than 1% of the hydrogen peroxide was utilized, the disinfectant properties of the hydrogen peroxide would disappear. In those circumstances where greater than 6% of the hydrogen peroxide was used, the conventional negative effects from high concentrations of hydrogen peroxide appeared, such as skin bleaching and delayed healing. In all circumstances and examples of the present invention, the remainder of the composition would be water. Typically, the hydrogen peroxide was of a quality that could be purchased over-the-counter. As such, the over-the-counter hydrogen peroxide will typically contain 3% hydrogen peroxide with the remainder being water. The water in the hydrogen peroxide solution is used to form the remainder of the composition when the smectite gelling agent is incorporated therein.

In experiments conducted with the present invention, the smectite gelling agent is a synthetic aluminosilicate having the formula (Na)ₓ(Al₂—XMgₓ)2Si₂O₁₀(OH)₂.n(H₂O) where X is between 0.1 and 0.5. Alternatively, the smectite gelling agent can be a synthetic aluminosilicate having the formula Naₓ(Mg₃—XLiₓ)3Si₄O₁₀(OH)₂ where X is between 0.1 and 0.5. The hydroxyl group (OH) can be substituted wholly or partially with a fluoride ion.

In the process of the present invention, the smectite gelling agent will be mixed with hydrogen peroxide and water together in a mixer. Under all circumstances, this mixture will be free of heavy metals. In particular, the mixer can be a tumbling closed container, a rolling closed container, a cowl dissolver, a paddle mixer, a colloid mill, and ultrasonicification mixer or combinations thereof. The hydrogen peroxide is mixed with water prior to the step of mixing the smectite gelling agent with the hydrogen peroxide. The smectite gelling agent of the present invention is formed from LAPONITE™. Laponite is a synthetic smectite clay that has many technological applications which go beyond the conventional uses of clays in pharmaceuticals and cosmetics. In biomedical applications, particularly in nanomedicine, LAPONITE™ has great potential. LAPONITE™ is a two-dimensional nanomaterial composed of disk-shaped nanoscale crystals that have a high-aspect ratio. These disks can strongly interact with many types of chemical entities (from small molecules or ions, to natural or synthetic polymers, or to different inorganic nanoparticles) and can also be easily functionalized and readily degraded in the physiological environment to the rise to non-toxic and even bioactive products.

In the present invention, it was unexpectedly discovered that the gel, composed of a synthetic silicate nanoparticle and an oxidizing aqueous agent and selected water-dispersible polymers will form a stable gel that will disinfect minor wounds of the skin and accelerate healing and self-assembly into a nanocomposite barrier film that protects the wound while healing occurs. This silicate nanoparticle appears to unexpectedly interfere with messenger molecules that initiate inflammation of the wound. This inflammation adds about three extra days to the healing process. The oxidizing media disinfects the wound so as to negate the need for the inflammation step. Further, the silicate self-assembles with the selected water-dispersible polymer to form a highly-ordered nanocomposite film that is impermeable to oxygen and bacteria.

As such, the present invention is a gel composed of a synthetic silicate of high purity (having no transition metal impurities) dispersed in an aqueous oxidizing agent and selected polymers that can be used to disinfect minor wounds to the skin, accelerate healing, and form a protective self-assembly nanocomposite barrier film that protects the wound during healing. The silicate is a synthetic aluminosilicate, such as “LAPONITE”™. The aqueous oxidizing agent can be 1-6% w/w hydrogen peroxide or 1-5% w/w providone-iodine. The water dispersible polymer can be polyvinylpyrrolidone, polyvinyl alcohol, polyvinyl acetate, polyethylene oxide, and natural polysaccharides, such as Gum Arabic. The polymer would be between 0.1-5% w/w of the composition.

The foregoing disclosure and description of the invention is illustrative and explanatory thereof. Various changes in the details of the described process or in the components of the described composition can be made within the scope of the present invention without departing from the true spirit of the invention. The present invention should only be limited by the following claims and their legal equivalents.

We claim:

1. A process for use in wound healing, the process comprising:
mixing a smectite gelling agent in a mixer with water and hydrogen peroxide to form a mixture, wherein the mixture is free of heavy metals; and
applying the mixture to the wound.

2. The process of claim 1, wherein the mixer is selected from the group consisting of a tumbling closed container, a rolling closed container, a cowl dissolver, a paddle mixer, a colloid mill, an ultrasonification mixer and combinations thereof.

3. The process of claim 1, the step of mixing comprising:
mixing the hydrogen peroxide with the water prior to the step of mixing the smectite gelling agent with hydrogen peroxide.

4. The process of claim 3, wherein the hydrogen peroxide is mixed in an amount of 1-6% w/w with the water.

5. The process of claim 4, wherein the hydrogen peroxide is mixed between 2-5% w/w with the water.

6. The process of claim 1, wherein the smectite gelling agent is a synthetic aluminosilicate having the formula (Na)ₓ(Al₂—XMgₓ)2Si₂O₁₀(OH)₂.n(H₂O), where X is between 0.1 and 0.5.

7. The process of claim 1, wherein the smectite gelling agent is a synthetic aluminosilicate having the formula Naₓ(Mg₃—XLiₓ)3Si₄O₁₀(OH)₂, where X is between 0.1 and 0.5.

8. The process of claim +, wherein (OH) is substituted wholly or partially with a fluoride ion.

9. The process of claim 7, wherein (OH) is substituted wholly or partially with a fluoride ion.

10. The process of claim 1, wherein the smectite gelling agent has a concentration between 1 and 7% w/w of the mixture.

11. The process of claim 10, wherein the smectite gelling agent has a concentration between 2 and 3% w/w of the mixture.

12. A composition for use in wound healing comprising:
a smectite gelling agent free of heavy and transition metals;
a hydrogen peroxide mixed with the smectite gelling agent; and
water.

13. The composition of claim 12, wherein the hydrogen peroxide is 1-6% w/w of the total composition.

14. The composition of claim 13, wherein the hydrogen peroxide is between 2-5% w/w of the total composition.

15. The composition of claim 12, wherein the smectite gelling agent is a synthetic aluminosilicate having the formula (Na)ₓ(Al₂—XMgₓ)2Si₂O₁₀(OH)₂.n(H₂O) where X is between 0.1 and 0.5.

16. The composition of claim 12, wherein the smectite gelling agent is a synthetic aluminosilicate having the formula Naₓ(Mg₃—XLiₓ)3Si₄O₁₀(OH)₂where X is between 0.1 and 0.5.

17. The composition of claim 15, wherein (OH) is substituted wholly or partially with a fluoride ion.

18. The composition of claim 16, wherein (OH) is substituted wholly or partially with a fluoride ion.

19. The composition of claim 12, wherein the smectite gelling agent has a concentration between 1-7% w/w of the total composition.

20. The composition of claim 19, wherein the smectite gelling agent has a concentration between 2-3% w/w of the total composition.
